(19) United States
(12) Patent Application Publication
(10) Pub. No.: US 2023/0079415 A1
(43) Pub. Date: Mar. 16, 2023

(54) EVAPORATIVE COOLING PAD SLEEP AID

(71) Applicant: Tyrone Dunham, Desert Hot Springs, CA (US)

(72) Inventor: Tyrone Dunham, Desert Hot Springs, CA (US)

(21) Appl. No.: 17/659,080

(22) Filed: Apr. 13, 2022

Related U.S. Application Data
(60) Provisional application No. 63/261,128, filed on Sep. 13, 2021.

(51) Int. Cl. A61F 7/02

(52) U.S. Cl. CPC A61F 7/02; A61F 2007/0058

(57) ABSTRACT

A flexible evaporative cooling pad includes a fabric base and at least two rows of fabric chambers affixed to the base. Each row of fabric chambers includes a first hydrogel-containing fabric chamber and an adjacent hydrogel-containing fabric chamber. Stitching isolates the hydrogel in each hydrogel-containing fabric chamber. The cooling pad cools under the legs or the head and neck and promotes the sleep cycle. In addition, the cooling pad may be used to treat headaches, fever, and hot flashes or just to cool down when it is hot. Use of an evaporative cooling pad may reduce the demand for air conditioning.

### CROSS-REFERENCE TO RELATED APPLICATION

This application claims the benefit of priority of U.S. provisional application number 63/261,128, filed Sep. 13, 2021, the contents of which are herein incorporated by reference.

### BACKGROUND OF THE INVENTION

The present invention relates to sleep aids and, more particularly, to an evaporative cooling pad sleep aid.

Adequate sleep is essential to one's health and wellbeing. Moreover, performance is adversely affected by a lack of sleep. The United States Centers for Disease Control and Prevention (US CDC) says that ⅓ of the adults in the US get less than the recommended amount of sleep and about half of adults experience insomnia at some time in their lives. The brain needs a slight drop in temperature before one can sleep. Everyone has different body temperatures and overheating seems to become an issue while sleeping. Hot leg syndrome and overheating interfere with falling asleep and staying asleep.

Currently available cooling pads are unsatisfactory for improving sleep. Pads with chambers that are “quilted” or single chambered, i.e., a gel filled single layer, do not have sufficient surface area exposure to make them effective and do not have the flexibility to make them comfortable. They absorb heat and are slow to cool back down, requiring refrigeration to work best. The lower surface area in a commercially available flat style pad reduces evaporative function and results in less cooling. They usually also have no moisture barrier base. Pads with no moisture barrier leave the surfaces moist and moisture is lost through the backing during use, leaving sheets clammy. Plastic packets need to be covered with fabric to be comfortable and are not easily washable.

As can be seen, there is a need for a cooling device that is flexible, has maximal surface area for evaporation, and has a moisture barrier base.

### SUMMARY OF THE INVENTION

In one aspect of the present invention, a flexible evaporative cooling pad comprises a fabric base and at least two rows of fabric chambers, affixed to the base, comprising a first hydrogel-containing fabric chamber, an adjacent hydrogel-containing fabric chamber, and stitching that isolates hydrogel in the first hydrogel-containing fabric chamber from hydrogel in the adjacent hydrogel-containing fabric chamber.

The inventive cooling pad provides cooling under the legs or head and neck and promotes the sleep cycle. In addition, the inventive cooling pad may be used to treat headaches, fever, and hot flashes or just to cool down when it is hot. Use of an evaporative cooling pad may reduce the demand for air conditioning.

These and other features, aspects and advantages of the present invention will become better understood with reference to the following drawings, description, and claims.

### BRIEF DESCRIPTION OF THE DRAWINGS

FIG. 1 is a perspective view of an evaporative cooling pad sleep aid according to an embodiment of the present invention;

FIG. 2 is a top plan view thereof;

FIG. 3 is a bottom plan view thereof;

FIG. 4 is a side elevation view thereof;

FIG. 5 is a detail cross-sectional view thereof, taken along line 5-5 of FIG. 4;

FIG. 6 is a detail cross-sectional view thereof, taken along line 6-6 of FIG. 5;

FIG. 7 is a top plan view thereof, shown in use;

FIG. 8 is another top plan view thereof, shown in use; and

FIG. 9 is a front perspective view thereof, shown in use.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, one embodiment of the present invention is a flexible evaporative cooling pad with rows of hydrogel chambers sewn to a moisture barrier fabric base, each pair of hydrogel-containing fabric chambers having stitching extending along a central longitudinal axis thereof. The stitching isolates hydrogel in each hydrogel-containing fabric chamber from hydrogel in an adjacent hydrogel-containing fabric chamber. The rows of paired hydrogel-containing chambers maximize surface area for evaporative cooling.

A base or backing formed of two base panels with silicone adhesive therebetween provides a moisture barrier that keeps the moisture from being pushed out of the pad and through the backing during use. In some embodiments, alternative materials may obviate the moisture barrier. The base may lack a moisture layer for pads intended for use in areas that do not come into contact with bedding and the like.

A plurality of dual hydrogel chambers may be centrally longitudinally sewn to the moisture barrier base in rows, maximizing surface area exposure for evaporative cooling. The centrally sewn dual rows of hydrogel chambers also provide flexibility during use. In some embodiments, the hydrogel-containing chambers may be affixed to one surface of the base.

In some embodiments, the hydrogel-containing chambers may be affixed to two surfaces of the base.

The dual rows of hydrogel chambers may be saturated with water by soaking. The polymer gel releases the water slowly and provides moisture to the fabric surface for evaporation and cooling. The hydrogel polymer also absorbs and holds heat quite well. The pad cools without refrigeration or electric power. Once the pad is hydrated by soaking in water or laundering in a washing machine, it stays hydrated for days. The user may use the pad under the legs at the end of the bed while trying to go to sleep or under the head and neck on the pillow to help induce the sleep cycle by lowering the temperature of the blood going to the brain. The pad may be restored by washing in a washing machine and may be used to cool the body for over a week at a time.

The materials of manufacture are not particularly limited. The base and chambers may be formed of any suitable fabric, such as high-wicking polyester and polycotton, i.e., a polyester-cotton blend. The base may comprise a silicone adhesive layer between two fabric panels to provide a moisture barrier. Each chamber may be filled with a specific dose of hydrophilic polymer gel.

The pad may be made of fabric using a sewing machine. The dual row chambers may be made from fabric strips folded lengthwise to the middle and sewn to form, for example, about 20″ long and about 2″ wide strips of fabric containing about eight about 4″ long and about 1″ wide chambers. The number and size of the fabric strips and thus the chambers may be optimized to improve cooling performance for specific applications. An upper rectangular fabric panel and a lower rectangular fabric panel having a length of about 20″ may be adhered together using a smoothing device by a silicone adhesive, e.g., about 5 to about 10 ounces, applied to the bottom surface of an upper rectangular fabric panel and the top surface of a lower rectangular fabric panel to form a base. The about five strips of chambers may be sewn to the upper fabric panel. One or more rows of stitching may be applied longitudinally down the centers of the fabric strips. Preferably, a polyacrylamide (PAM) polymer is precisely dosed with about 2 to about 2.5 grains per inch to the chambers, for example about eight 4-inch long 1-inch-wide chambers with about 8 grains to about 10 grains of polymer in the 4-inch-long chambers. For example, the pad may include about 320 to about 400 grains of potassium polyacrylamide (KPAM). Two about 15″ and two about 21″ binding strips may be sewn around the edges.

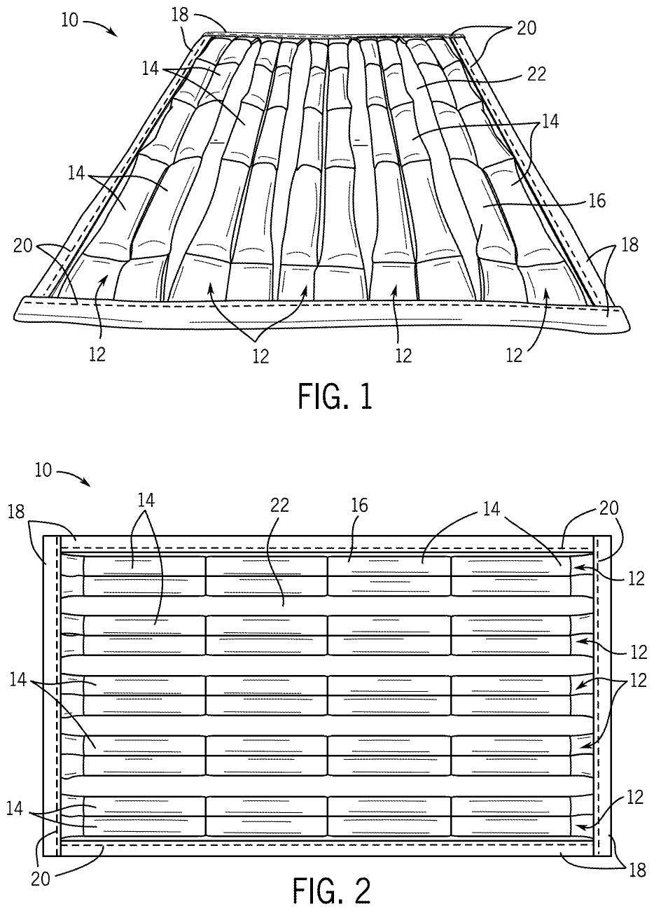

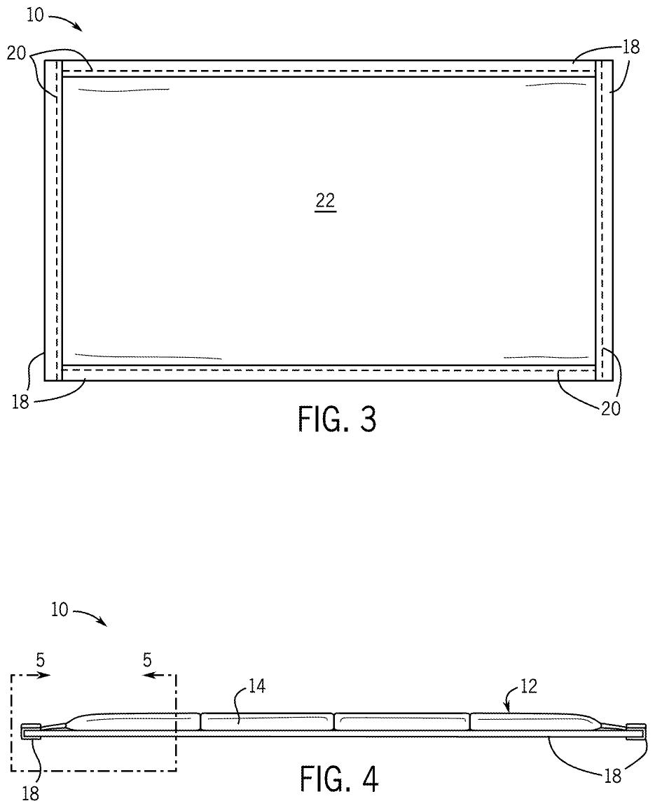

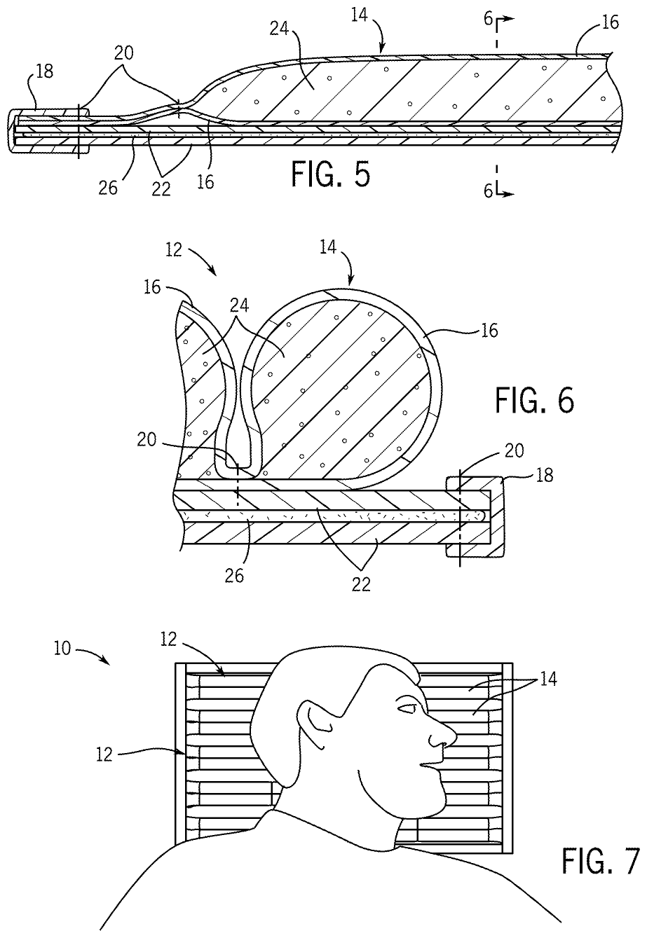

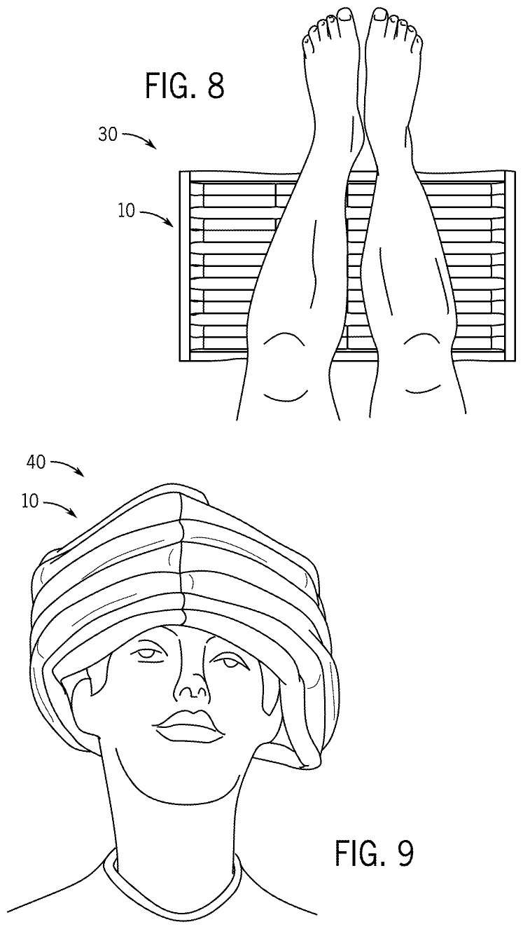

Referring to FIGS. 1 through 9, a cooling pad 10 is shown with several rows of dual hydrogel chambers 12. A row of hydrogel chambers 12 comprises two hydrogel-containing chambers 14. The cooling pad comprises polycotton fabric 16 having a binding of edging material 18 attached with stitching 20 to a moisture barrier base material 22. As illustrated in FIGS. 5 and 6, the chambers 14 contain a hydrated polymer 24 which slowly releases water for evaporative cooling. The moisture barrier base comprises two layers of moisture barrier base material 22 coupled by silicone adhesive 26. The cooling pad 10 may be used in several ways as seen in FIGS. 7 through 9, such as under the head, under the legs 30, and wrapped around the head 40.

It should be understood, of course, that the foregoing relates to exemplary embodiments of the invention and that modifications may be made without departing from the spirit and scope of the invention as set forth in the following claims.

What is claimed is:

1. A flexible evaporative cooling pad comprising:
a) a fabric base; and
b) at least two rows of hydrogel-containing fabric chambers, affixed to the fabric base, each row comprising a first hydrogel-containing fabric chamber, an adjacent hydrogel-containing fabric chamber, and stitching that isolates hydrogel in the first hydrogel-containing fabric chamber from hydrogel in the adjacent hydrogel-containing fabric chamber.

2. The flexible evaporative cooling pad of claim 1, wherein the fabric base comprises two fabric panels adhered together with silicone adhesive.

3. The flexible evaporative cooling pad of claim 1, wherein the stitching extends along a central longitudinal axis of the at least two rows of hydrogel-containing fabric chambers.

4. The flexible evaporative cooling pad of claim 1, wherein the at least two rows of hydrogel-containing fabric chambers is affixed to one surface of the fabric base.

5. The flexible evaporative cooling pad of claim 1, wherein the at least two rows of hydrogel-containing fabric chambers is a plurality of fabric chambers affixed to two surfaces of the fabric base.

6. The flexible evaporative cooling pad of claim 1, wherein the fabric base comprises a silicone adhesive layer coupling two fabric panels.

7. The flexible evaporative cooling pad of claim 1, wherein the first hydrogel-containing fabric chamber has isolated chamber segments and the adjacent hydrogel-containing fabric chamber has isolated chamber segments.

8. The flexible evaporative cooling pad of claim 1, wherein the first hydrogel-containing fabric chamber and the adjacent hydrogel-containing fabric chamber contain about 2 grains to about 2.5 grains of potassium polyacrylamide per square inch.

9. The flexible evaporative cooling pad of claim 1, further comprising a fabric binding along edges of the fabric base.

10. The flexible evaporative cooling pad of claim 1, wherein the fabric base is rectangular.
